(19) United States
(12) Patent Application Publication
(10) Pub. No.: US 2013/0289546 A1
(43) Pub. Date: Oct. 31, 2013

(54) PRODUCING HIGHER OPTICAL ABLATIVE POWER USING MULTIPLE PULSES HAVING CONTROLLABLE TEMPORAL RELATIONSHIPS

(71) Applicants: RICHARD STOLTZ, Plano, TX (US); Jeff Bullington, Chuluota, FL (US)

(72) Inventors: RICHARD STOLTZ, Plano, TX (US); Jeff Bullington, Chuluota, FL (US)

(21) Appl. No.: 13/632,032

(22) Filed: Sep. 30, 2012

Related U.S. Application Data
(60) Provisional application No. 61/542,909, filed on Oct. 4, 2011.

(51) Int. Cl. A61B 18/20

(52) U.S. Cl. 606/11

(57) ABSTRACT

The use of multiple pulses having controllable temporal relationships can produce higher optical ablative pulse peak power using split by color pulses. Split pulses can be controllably delayed to increase pulse peak power, average power and/or repetition rate in single-amplifier or multi-amplifier systems.

### CROSS-REFERENCE TO RELATED APPLICATION

This is a continuation-in-part application of and claims the benefit of pending U.S. Provisional Application No. 61/542,909 filed Oct. 4, 2011 which is hereby incorporated herein by reference.

### BACKGROUND OF THE INVENTION

This invention relates to a method of laser ablation of material. Laser ablation of material and the creation of plasma are very complex processes. The removal of atoms from the bulk material is done by vaporization of the bulk at the surface region in a state of non-equilibrium and is caused by a coulomb explosion generated from multi-photon ionization created by the peak power in the laser pulse. The incident laser pulse penetrates into the surface of the material within the penetration depth. This dimension is dependent on the laser wavelength and the index of refraction of the target material at the applied laser wavelength and is typically in the region of 10 nm for most materials. The strong electrical field generated by the laser light is sufficient to remove/ionize the electrons from the bulk material. This process is initiated through the peak power in a laser pulse and is caused by non-linear multi-photon ionization. The free electrons oscillate within the electromagnetic field of the laser light and can collide with the atoms of the bulk material thus transferring some of their energy to the lattice of the target material within the surface region resulting in the material vaporizing and being ejected in an ionization plume.

To achieve ablative pulse peak power while getting good energy efficiency, the ablation pulses should be short, preferably less than about 10 picosecond.

A typical ablation system uses about 1 picosecond or shorter pulses, as longer duration pulses are much less efficient; and typically use optical compressors that have a maximum time compression that temporally shortens an amplified pulse length from about 1 nanosecond (ns) to about 10 picosecond (ps). Initial pulses ablate a body surface and a freshly uncovered surface then gets ablated by a subsequent pulse. This can continue to make a deep hole, or a hole through the material, or a cut into or through a material.

This can be done with light in infrared, visible and ultraviolet, and light can be described by its wavelength, frequency or color. For ease of description, even in the infrared and ultraviolet comparisons of wavelengths are often described as colors, with redder being the longer wavelengths and bluer being the shorter wavelengths.

The pulse generator generates a ramp of wavelengths, e.g. from bluer to redder over a 1 nanosecond period of time, and this ramp allows the pulse to be compressed in time by the optical compressor.

Prior art U.S. Pat. No. 7,361,171 by Richard Stoltz and Jeff Bullington (the present inventors) discloses an apparatus and method of surgical ablative material removal “in-vivo” or from an outside surface with a short optical pulse that is amplified and compressed using either an optically-pumped-amplifier and air-path between gratings compressor combination or a semiconductor optical amplifier (SOA) and chirped fiber compressor combination, wherein the generating, amplifying and compressing are done within a man-portable system.

That prior art also discloses a method of ablative material removal from a surface of a body, comprising the steps of: generating an initial pulse in a pulse generator; amplifying the initial pulse using an amplifier comprising either an optically-pumped-amplifier or a semiconductor optical amplifier within a man-portable system, to generate an amplified pulse; compressing the amplified pulse within the man-portable system using a compressor, to generate a compressed optical pulse having a duration of one picosecond or less; and applying the compressed optical pulse to the surface of the body, wherein the generating, amplifying and compressing steps are performed within the man-portable system, and wherein more than one amplifier is used in a train mode to amplify the initial pulse.

### SUMMARY OF THE INVENTION

Ultra-short pulse lasers use light at high power to precisely remove material by ablation, essentially without heating the workpiece. An ablation pulse is a laser pulse having peak power which causes non-linear multi-photon ionization with a strong electrical field generated by the laser light that is sufficient to remove electrons from the bulk material, resulting in the material vaporizing and being ejected in an ionization plume.

Such systems use an optical pulse generator, a series of optical amplifiers, and an optical pulse compressor. A typical ablation system uses about 10 picosecond or less long pulses, as longer duration pulses are much less efficient; and typically use optical compressors that have a maximum time compression that temporally shortens an amplified pulse length from about 1 nanosecond (ns) to about 1 picosecond (ps). The pulse generator generates a ramp of wavelengths, e.g. from bluer to redder or from red to blue over a 1 nanosecond period of time, and this ramp allows the pulse to be compressed in time by the optical compressor. Portable systems generally use glass-fiber amplifiers. Portable ultra short pulse laser ablation systems typically use an amplified pulse (e.g. from a series of fiber amplifiers) use a stretched pulse with a maximum stretch of about one nanosecond (1 ns) in length, followed by an optical pulse compressor which compresses the pulse to about ten picosecond (10 ps) or less (which is generally the desired pulse length for laser ablation).

Such systems are generally limited by the maximum peak-power limitation of their optical amplifiers.

The novel method herein avoids peak power limitations in fiber amplifiers by enabling the use of a stretched pulse longer than 1 ns, splitting the pulse by color into portions with different paths after amplification and then realigning the paths to be compressed and then outputted as a single ablation beam. Further, as the frequencies in the portions are different, the realigned portions can be overlapped without optical interference and then compressed. By controlling the path lengths of the portions between when the portions are split apart and when they are realigned, the timing of the portions can be controlled.

As the pulse is split, realigned, and compressed using grating compressors in air, the peak power of the final pulse is not limited by the peak power limitation of optical fiber.

Preferably, the portions can be stacked for higher output pulse power, or offset in time to give ablation pulses spaced at less than picoseconds apart for higher efficiency. In either case, the (e.g. 1 ns) portions generally overlap when realigned before going into the compressor, but in the case of the spaced output pulse, it comes out of the compressor as two (e.g. 1 ps) ablation pulses.

Preferably a dichroic mirror is used to reorient pulses into a single beam in air going into the compressor.

In some embodiments, a dichroic mirror is used to split amplified pulses.

In some embodiments, pulses are amplified longer (e.g., higher energy), and then the amplified pulses are split by color pulses into pieces with lengths that can be compressed to about 10 picosecond or less, and thus effectively be used for ablation.

A present limitation in the technology is to have a low loss compact (portable) optical compressor. If small size compressors with greater compression become available; the improvements herein can be applied to those as well, with even longer pulses being split by color.

This can be a method of ablative material removal from a surface of a body, which includes the steps of: generating an initial pulse in a pulse generator; amplifying the initial pulse using an optically-pumped, optical amplifier within a portable system, to generate an amplified pulse; then splitting the amplified pulse into at least two split pulses, which at least two split pulses have different optical paths, and controlling optical path lengths of said at least two split pulses to determine a temporal relationship between said two split pulses; then reorienting said at least two split pulses on a common path in air; then compressing the amplified pulse within the portable system using at least one air-path between gratings optical compressor, compressing the first and second amplifier pulses using a compressor to produce an ablation pulse output; and applying the compressed optical pulse to the surface of the body, wherein the generating, amplifying, and compressing steps are performed within the portable system.

With the reorienting of the at least two split pulses on a common path being an air path, the realigned portions can be overlapped without optical interference (caused, e.g., by out of phase wavelengths in the same place at the same time), and the peak power is not limited by the peak power limitation of optical fiber. Note the compressing is in an air-path between gratings compressor and likewise and the peak power limitation of optical fiber is not a problem and the different wavelengths of the split pulses avoid optical interference in the compressor as well.

Preferably, the splitting and rejoining are done with dichroic mirrors and the controlling of optical path lengths of said at least two split pulses to determine a temporal relationship between said two split pulses is done by spacing of full reflecting mirrors and dichroic mirrors. While there are other methods of splitting and rejoining optical pulses, dichroic mirrors are preferred, especially for the rejoining.

The controlled pulse paths between the splitting and rejoining dichroic mirrors can be done with reflecting mirrors in air or in optical fiber, or a combination or the two.

A system can use an e.g. 2 ns (nanosecond) amplified pulse that is split into two pulses with controlled optical path lengths between splitting and reorienting said two split pulses on a common path then using a normal compression ratio to achieve approximately double output average power and the repetition rate at a relatively small additional system cost. Or, by stacking the pulses in time, it can substantially double the pulse peak power.

Splitting and rejoining of two pulses can be cost effective, as much of the system remains the same and the relatively small additional system cost is much less than the cost of an additional system.

Splitting and rejoining the more than two pulses can be even more cost effective. Dichroic mirrors can be used to provide additional splitting, e.g. the redder portion can be split into a smaller-“reddest” part and a smaller-“not-quite-as-red” part; and bluer portion can be split into a smaller-“bluest” part and a smaller-“not-quite-as-blue” part. The four parts can be combined to, approximately quadruple output average power and, by controlling path lengths, e.g., double the repetition rate and substantially double the pulse power, all at a reasonably small additional system cost.

Further, these parts can be cut by color into even more pieces. It should be noted that additional pumping of the amplifiers is generally needed.

This can also be a method of ablative material removal from a surface of a body, comprising: generating initial pulses in a pulse generator; obtaining at least two pulses having different wavelengths into different optical paths; amplifying the obtained pulses using separate optically-pumped optical amplifiers to generate separate amplified pulses; reorienting said separate pulses onto a common path in air using a dichroic mirror, while controlling optical path lengths of said separate pulses to determine a temporal pulse relationship to form at least one reoriented pulse; Then compressing the at least one reoriented pulse within the portable system using an air-path between gratings compressor, to generate at least one compressed optical ablation pulse; and applying the compressed optical pulse to the surface of the body, wherein the generating, amplifying and compressing steps are performed within the portable system.

In some embodiments, more than one initial pulse having different wavelengths are generated by the pulse generator and at least two pulse pickers are used to get pulses into having different optical paths.

In some embodiments, the at least two split pulses are overlapped during reorienting to produce a single ablation pulse.

In some embodiments, the at least two split pulses are offset-overlapped during reorienting to produce a two ablation pulses.

The controlling optical path lengths of said at least two split pulses to determine a temporal relationship between said two split pulses can be done using optical delay trimmer devices and the placement of mirrors in optical paths. Optical fibers can be used with or instead of mirrors.

In the embodiment where parallel amplifiers are used between the splitting and the reorienting, optical delay devices and the placement of mirrors in optical paths are also used, but also controlled links fiber can be used and, further, the lengths of fiber in the amplifiers should also be taken in consideration.

This can alternately be a method of ablative material removal from a body, comprising: generating an initial pulse in a pulse generator; amplifying the initial pulse using of two optical amplifiers in series within a portable system, wherein two amplifiers are used to generate an amplified pulse, wherein the first amplifier is an optically pumped fiber-amplifier and a second amplifier is a Raman amplifier; then splitting the amplified pulse into at least two split pulses having different optical paths, and controlling optical path lengths of said at least two split pulses to determine a temporal relationship between said two split pulses; then reorienting said at least two split pulses on a common path in air; compressing the at least two split pulses using at least one air-path between gratings compressor to produce at least one ablation pulse output; and applying the at least one compressed optical ablation pulse to the body, wherein the generating, amplifying and compressing steps are performed within the portable system; and controlling optical path length between said initial pulse splitter and said compressor to determine a temporal relationship between said first and second amplifier output pulses, whereby at least one of pulse energy and repetition rate can be at least substantially doubled, whereby at least one of pulse energy and repetition rate can be at least substantially doubled.

As a given compressor has a predetermined temporal compression ratio, running the amplifier with a longer pulse length in the amplification achieves a higher energy and average power in the pulse with no nonlinear effects, and then splitting the single pulse after amplification into pulses by color (wavelength/frequency) provides reduction of length of split pulse (dichroic mirrors or filters) where each separate pulse has a fraction of the temporal length of the original with each pulse having sufficient bandwidth to enable the compression to the predetermined temporal compression ratio; thus the system with a pulse stretched to two to three times the normal compression ratio can achieve output powers with double or triple the repetition rate and average power or, by stacking the pulses in time, double, triple, or more the pulse energy; as compared to the normal systems using the same amplifiers. In this technology additional care needs to be provided to preserve and generate the bandwidth with gain flattening filters before gain stages.

Splitting a long-amplified pulse by color/frequency into at least two pulses can be used in combination with any of the configurations below:

A long stretch chirped pulse is able to extract more energy without reaching the peak power limitation that induces non-linear effects in the gain media. The long stretch chirped pulse simplifying the amplifier chain and control systems resulting in lower cost and higher performance. Long stretched chirped pulses past a 1 ns stretch is difficult to compress do to the grating size for a single pass compression element or the high optical losses and optical complexity for a multi-pass compression element. Hence, chirp pulse amplifier (CPA) systems rarely utilize pulses lengths greater than 1 ns independent of the other system level benefits. The longer stretched chirped pulse may require some additional pulse conditioning to preserve the generated bandwidth, by use of gain flattening filter or pulse shaping before the gain stages. After storing the maximum energy in the long stretched chirped pulse where the pulse has achieved a peak power just below the non-linear threshold of the gain media the pulse is split into multiple pulses. This separates the original pulse into two pulses where each pulse has half of the temporal pulsewidth and half of the frequency/color bandwidth of the original pulse. Utilizing a dichroic mirror or filter to split the original optical pulse minimizes the loss of optical power. The original pulse bandwidth was generated and maintained to produce two separate pulses with sufficient bandwidth to be compressed to the desired pulsewidth utilizing a predetermined temporal compression ratio; thus the system with a pulse stretched to two times the normal compression ratio can achieve output powers with double the repetition rate/average power or, by stacking the pulses in time, double the pulse energy; as compared to the normal systems using the same amplifiers with a predetermined temporal compression ratio.

Amplifying of a pulse for longer than its nominal amplification time can get an amplifier output energy pulse higher than the nominal amplifier output pulse energy. The optical output can have at least one of pulse energy and repetition rate that is at least substantially doubled as compared to its nominal value.

Another example of this technology can be seen using an optical pulse with 20 nm of bandwidth with an initial pulse width of 100 fs of bandwidth center at 1 μm can be stretched using dispersion compensating fiber or other method to a 4 ns pulse length. The 4 ns pulse shape may require pulse shaping such as quadratic pulse shaping to preserve the bandwidth during amplification. The 4 ns pulse allows the use of less expensive fiber amplifier chain and achieves a greater amount of energy in the pulse relative to a 1 ns pulse. After amplification the 4 ns pulse can be split using dichroic beam splitters and mirrors into 4 separate 1 ns pulses, each with 5 nm of bandwidth. The position of each pulse can be modified by the relative position of the dichroic beam splitters and mirrors so the pulses can overlap temporally or be spaced at an arbitrary time spacing. Each 5 nm 1 ns pulse can be compressed using a Tracey grating compressor to the sub 10 ps level.

Ultra short pulse laser ablation systems typically use an amplified pulse (e.g. from a series of fiber amplifiers) use a stretched pulse with a maximum stretch of about one nanosecond (1 ns) in length, followed by an optical pulse compressor which compresses the pulse to about one picosecond (1 ps) or less (which is generally the desired pulse length for laser ablation). The limitation in the technology is to have a low loss compact optical compressor. Small size compressors with greater compression cannot be manufactured; the improvements herein can be applied to those as well.

Thus, using small-size compression of, e.g. one ns to one ps and the maximum peak power has limited the ablation rate. Using more than one laser for ablating a work-peace generally causes beam alignment problems and can cause optical interference.

Here, longer than 1 ns amplified pulses can be cut into smaller, e.g. 2 or more 1 ns portions, allowing stacking into higher power pulses (or more pulses) while avoiding beam alignment problems and optical interference. Thus a 3 ns pulse may be cut into 3 one-ns pieces; a 4 ns pulse into 4 one-ns pieces, etc. by adding more dichroic and regular mirrors.

More effective ablation can be achieved by either higher pulse power or more pulses which can be closely place in time (e.g. less than about 20 ps apart). Further the techniques herein can also be used to increase (double, triple, etc.) the system pulse repetition rate and thus increase the average power.

The amplifier input and the amplified pulse both have a varying frequency, swept with time (e.g. from lower frequency to higher frequency), and that variation allows pulses to be compressed, and to be cut in pieces e.g. by a dichroic mirror.

As shown in the figures, splitting pulses is made possible by the varying of frequency within the pulse. The splitting of pulses into at least two pieces is preferably by dichroic mirrors which can be used to reflect the redder frequencies while transmitting the bluer frequencies. By controlling the path lengths, the timing of the portions can be controlled. As the frequencies in the portions are different, the portions can be overlapped without optical interference.

### BRIEF DESCRIPTION OF THE DRAWINGS

For a better understanding of the invention reference may be made to the accompanying drawings. The invention can function with light in infrared, visible and ultraviolet, and light can be described by its wavelength, frequency or color. For ease of description, colors are described in the drawings indicated with an “R” redder (longer wavelength) and “B” as bluer (shorter wavelength) and “R” and “B” both redder and bluer wavelengths.

FIG. 1, shows (using visible light colors comparison (redder and bluer), for demonstration, rather than the preferred infrared colors/(wavelength, e.g. centered around 1,550 nm) the use of a dichroic mirror to split, by color, a longer 2 ns pulse (e.g. from a high power fiber amplifier) into two 1 ns compressible-pulses;

FIG. 2, shows the use of a dichroic mirror to split, by color, a 2 ns pulse into two 1 ns compressible-pulses and providing a longer path length such that the redder pulse is separated in time from the bluer pulse and then realigning the pulses thus increasing system repetition-rate and power-output; and,

FIG. 3, shows the use of a dichroic mirror to split, by color, a 2 ns pulse into two 1 ns compressible-pulses and providing a longer path length for the bluer pulse such the redder pulse catches up in time and overlaps the bluer pulse to give a very high energy 1 ps pulse compressible pulses. The overlap can be a total overlap and a single ablation pulse output, or a slightly offset overlap, e.g. offset by 15 ps, to give two closely-spaced ablation pulses.

### DETAILED DESCRIPTION OF THE PREFERRED EMBODIMENTS

Ultra-short pulses of light at high power can precisely remove material by ablation, essentially without heating the workpiece. A typical ablation system uses about 1 picosecond long pulses, as longer duration pulses as less effective, and uses compressors that can (at maximum time compression) temporally shorten the pulse length from about 1 nanosecond to about 1 picosecond. Such a system also has glass-fiber amplifiers that have a peak-power limitation.

A system with an e.g. 2 ns long amplified pulse can be split into two or more pulses and then using a normal compression ratio can achieve output powers double or more the repetition rate/average power or, by stacking the pulses in time, double or more the pulse energy at a relatively low additional system cost.

Splitting pulses with a conventional splitter and staggering optical path delays of the pulses before introduction into two or more amplifiers can provide a higher system repetition rate/average power. This splitting occurs best prior to the last gain stage.

Using pulses from a common pulse generator that are transmitted into a multiple set of amplifiers, either in series or parallel, allows precision timing between pulses; controlling path lengths by adjusting length of optical fiber or time delays induced through mechanical, electrical, or thermally control elements in the low power portions of the system between pulse generator and different amplifiers can give nanosecond or even picosecond timing of output pulses; the delay can be induced in a single fiber for series amplifiers and in separate fibers for parallel amplifiers it also avoids a pulse during the plume of a previous ablation pulse (the plume can start as early as about 0.1 ns after an ablation pulse, and lasts about 100,000 ns, and can continue strong enough to cause significant inefficiencies.

Picosecond spacing of pulses can provide significantly more efficient machining. If split by color into three pulses, the use of a doubled-peak power pulse closely followed by the second pulse of single peak power in less than 0.01 ns (10 ps) can provide effective machining. It takes advantage of energy from the doubled-peak power pulse that is stored in the material being ablated that has not had time to dissipate. This can also be used as a longer-amplified pulse split by color single or multiple amplifier system.

Dividing pulses by color prior to amplification allows stacking of output pulses from parallel amplifiers which gives higher energy pulses. The pulses can be stacked without interference as they are at different wavelengths/frequencies, and stacked in air to avoid optical fiber peak power problems.

Controlling path lengths by adjusting length of optical fiber and using adjustable optical delays between pulse generator and different amplifiers can then be trimmed to sub-picosecond timing of pulses and provide precision stacking of pulses; thus providing higher pulse power. The same controlling of optical path length and then trimming technique can be used with splitting by color of amplified pulses.

Using four amplifiers in parallel in stacked-pulses series-pulses configuration can give a doubled pulse energy, doubled repetition rate system with four times the system output power.

Using four amplifiers in parallel in stacked-pulses series-pulses configurations together with, can give increased pulse energy, doubled repetition rate system with four times the system output power, in combination with splitting by color the four longer-amplified pulses into 2 or 3 pulses each can give 8 to 12 times the pulse energy, as compared to the normal systems using the same amplifiers.

Using more than four amplifiers in parallel with outputs configured to give stacked-pulses and series-pulses can give even higher pulse energies.

FIG. 1, demonstrates (using visible light colors, rather than the preferred infrared colors/wavelengths, e.g. centered around 1,550 nm) the use of a dichroic mirror 10 to split, by wavelength, a longer 2 ns pulse (e.g. from a high power fiber amplifier) into two 1 ns (compressible to 1 ps)-pulses; thus increasing system power output while maintaining compressible pulses and avoiding the amplifiers peak-power limitation and making the fiber amplifiers less expensive.

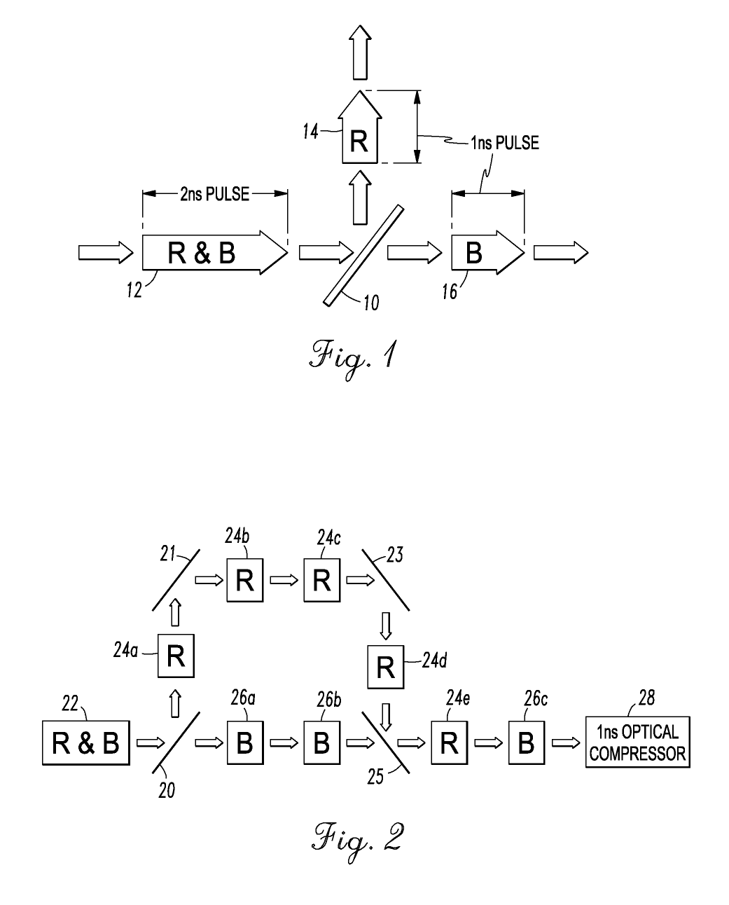

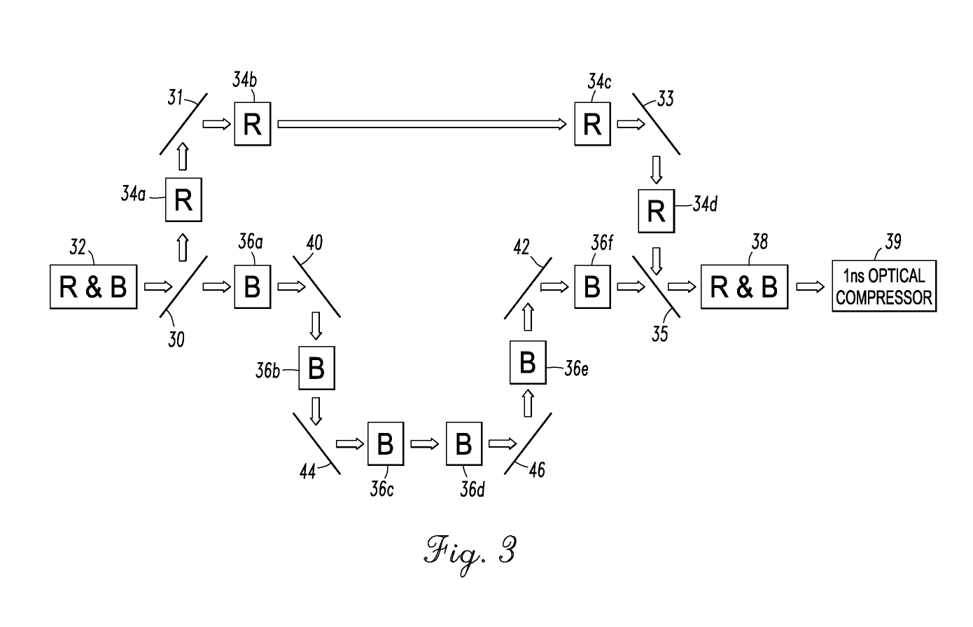

FIG. 2, demonstrates the use of a dichroic mirror 20 to split, by wavelength, with a longer e.g. 2 ns pulse 22 being split into two 1 ns compressible-pulses and providing a longer path length such that the redder pulse (24a, 24b, 24c, 24d, and 24e) is separated in time from the bluer pulse (26a, 26b, and 26c) and then realigning the pulses. Thus system repetition-rate and power-output can be increased while maintaining compressible pulses and avoiding the amplifiers peak-power limitation. The centerlines of the redder and bluer pulses are aligned and thus can be compressed in a single compressor 28 to two 1 ps pulses and outputted in a conventional output stage.

In FIG. 2, dichroic mirror DCM1 20 reflects the redder portion 24a and passes the bluer portion 26a. Mirrors M1 21 and M2 23 reflect the redder pulse 24b→24c→24d toward dichroic mirror DCM2 25 and provide a greater path length to delay the redder pulse. The dichroic mirror DCM2 25 reflects the redder 24d pulse and passes the bluer pulse 26c. The dichroic and conventional mirrors are configured to align the two output pulses.

FIG. 3, demonstrates the use of a dichroic mirror DCM1 30 to split, by wavelength, a 2 ns pulse into two 1 ns compressible-pulses and providing a longer path length for the bluer pulse such the redder pulse catches up in time and overlaps the bluer pulse to give a very high energy 1 ps pulse compressible pulses while avoiding the amplifier's peak-power limitation. As the colors of the redder and bluer pulses are different, interference between the overlapped pulses is avoided. The overlap can be a total overlap and a single ablation pulse output, or a slightly offset overlap, e.g. offset by 15 ps, to give two closely-spaced ablation pulses.

Two aligned closely spaced (e.g. a few picoseconds apart) 1 ps pulses can be more than 20% more efficient than two aligned pulses spaced tens of microseconds apart; thus even if the redder pulse has less energy than the bluer pulse it still can have close to the same ablation effect.

In FIG. 3, the 2 ns input pulse 32 to dichroic mirror DCM1 30 is split reflecting the redder portion 34a and passing the bluer portion 36a. Mirrors M1 31 and M2 33 reflect the redder pulse toward dichroic mirror DCM2 35 and provide a lesser part length for the redder pulse. Mirrors M3 40, M4 44, M5 46, and M6 42 reflect the bluer pulse toward dichroic mirror DCM2 35 and provide a greater path length to delay the bluer pulse. The dichroic mirror DCM2 35 reflects the redder pulse and passes the bluer pulse. The dichroic and conventional mirrors are configured to align the two output pulses.

If the splitting is before power amplification (not shown) and the redder and bluer portions are separately amplified in separate amplifiers, then the amplified pulses can be realigned. This, similar to the FIG. 3 configuration, can double the system power with the aligned closely spaced pulses more than doubling the ablation rate, an overlap can be included or a slightly offset overlap, e.g. offset by 15 ps, to give two closely-spaced ablation pulses.

Splitting into four pieces is similar; e.g. a first dichroic mirror can split one pulse into two halves, and a second dichroic mirror can be used to split a half into two quarters, and a third dichroic mirror can be used to split the other half into two more quarters, thus giving four pieces in four paths.

Reorienting four pulses in four paths into a single output beam is also similar; two dichroic mirrors can combine four paths into two paths, and another dichroic mirror can be used to combine those two paths into a single output beam.

The splitting before power amplification and the redder and bluer portions separately amplified in separate amplifiers, can double the system power and double the peak pulse power. The total overlap and a single ablation pulse output with twice the peak power.

Dichroic mirrors can similarly be use to split longer pulses into more pieces, e.g. three or more, and with two or more amplifiers make even higher repetition rates and/or even higher pulse energies.

In the figures the dichroic mirrors are used to reflect the redder frequencies while transmitting the bluer frequencies, thus the varying frequency allows the pulse to be split into two or more pulses. By controlling the path lengths, the timing of the portions can be controlled. Further, as the frequencies in the portions are different, the portions can be overlapped without optical interference.

In FIGS. 2 and 3, the portions are aligned along an optical axis for compression and can give an aligned output beam.

Note that in FIG. 2 the spacing of the uncompressed portions would give an increased system pulse repetition rate, but would not give the closely placed in time, less than about 20 ps apart pulses. The closely placed in time pulses going into the compressor would be mostly overlapping, just slightly offset (e.g. with a 10 or 20 ps offset or a 99% or 98% overlap), see FIG. 3 note that 99% or 98% overlaps are impractical to illustrate in a drawing.

FIG. 1 shows splitting by color with a dichroic mirror 10, reflecting the redder frequencies while transmitting the bluer frequencies of 2 ns pulse 12, to give redder 1 ns long portion 14, and bluer 1 ns long pulse 16.

FIG. 2 shows splitting by dichroic mirror DCM1 20, reflecting the redder frequencies of the 2 ns pulse 22 while transmitting the bluer frequencies of the 2 ns long pulse 22, to give redder 1 ns long portion 24a, and bluer 1 ns long portion 26a. The redder 1 ns long portion 24a is reflected off dichroic mirror DCM1 20 as portion 24b and which goes as portion 24c and is reflected off mirror M1 21, then goes as portion 24d and is reflected off mirror M2 23, then in rejoining, the portion 24d in air is reflected off dichroic mirror DCM2 25 towards the optical compressor 28 as portion 24e, also in air. The bluer 1 ns long portion 26a (the other portion of the 2 ns pulse 22) having been transmitted through dichroic mirror DCM1 20, then as 26b enters, and is transmitted through dichroic mirror DCM2 25 exiting as portion 26c in air.

Alternately (not shown) a digital phase modulator or pulse shaper such as a Dazler™, that uses a custom-optic, liquid crystal or other optical phase modulation technology, can be inserted between the dichroic mirror DCM2 25 exiting as portions 24e and 26c (in FIG. 3) to reduce higher order phase distortion thereby reducing optical losses in the compression of the pulse portions 24e and 26c. The portions are aligned along an optical axis for compression by the “1 ns Optical compressor” (which can compress each of the pulses from 1 ns to 1 ps) which then can give an aligned output beam. Here the bluer portion path is slightly shorter than the red path and the redder portion is delayed, (e.g. by 0.5 ns) to give separation between compressor input pulses.

Alternately (not shown in FIG. 2) the path lengths can be such that the pulses entering the compressor are partially overlapping but will exit the compressor as more closely spaced (e.g. by 10 ps) pulses.

FIG. 3 has a redder portion path generally the same as FIG. 2, but the bluer portion path is longer than the redder portion's path to delay the bluer portion by, (e.g. 1 ns), such that the two portions totally overlap, or are just slightly offset (e.g. with a 10 or 20 ps offset or a 99% or 98% overlap).

FIG. 3 shows dichroic mirror DCM1 30, reflecting the redder frequencies of the 2 ns pulse 32 to give portion redder portion 34a, while transmitting the bluer frequencies of the 2 ns long pulse 32, to give bluer 1 ns long portion 36a. Redder (1 ns long) portion 34a is reflected off mirror M1 31 to give portion 34b and which is then goes as portion 34c and is reflected off mirror M2 33, then as 34d is reflected off dichroic mirror DCM2 35 to become the red portion of pulse 38. The bluer (1 ns long) portion 36a having been transmitted through dichroic mirror DCM1 30, then as 36b is reflected off mirror M3 40, as portion 36b, which is reflected off mirror M4 40, as 36c, then as 36d is reflected off mirror M5 46, as 36e, which is reflected off mirror M6 42, as 36f, which enters and is transmitted through dichroic mirror DCM2 35 exits to become the blue portion of pulse 38. The combined portions are aligned along an optical axis for compression by the 1 ns Optical compressor 39 which then can give an aligned output beam.

The use of multiple pulses having controllable temporal relationships can produce higher optical ablative power using split pulses. Split pulses can be delayed and/or trimmed to increase pulse energy and/or repetition rate in single-amplifier or multi-amplifier systems.

In one embodiment, the amplifier is run longer than its nominal amplification time to get higher energy pulses and higher efficiency, and then split into pulses by color (wavelength) to provide reduction of length of split pulses and each pulse compressed (at the compressor predetermined temporal compression ratio) to obtain the ultra-short length of pulses needed for effective ablation; thus the system may have a factor of two times the output power with double the repetition rate or, by stacking the pulses in time, double or triple the pulse energy; as compared to the normal systems using the same amplifiers. Splitting a longer-amplified pulse by color into at least 2 pulses can be used in combination with any of the multi-amplifier configurations below.

The longer stretched chirped pulse (2 ns) may require some additional pulse conditioning to preserve the generated bandwidth, by use of gain flattening filter or pulse shaping before the gain stages. After storing the maximum energy in the long stretched chirped (2 ns) pulse where the pulse has achieved a peak power just below the non linear threshold of the gain media the pulse is split into multiple pulses. For a two pulse concept the original pulse is split into two pulses using a dichroic mirror or filter set at the center frequency of the original pulse. This separates the original pulse into two pulses where each pulse has half of the temporal pulsewidth and half of the frequency/color bandwidth of the original pulse (i.e.: A 2 ns pulse is separated into 2—1 ns pulse where each pulse can be compress using a 1 ns compressor element. The compression can be accomplished on pulses that are either temporally sequential or simultaneous. Utilizing a dichroic mirror or filter to split the original optical pulse minimizes the loss of optical power. The original pulse bandwidth was generated and maintained to produce two separate pulses with sufficient bandwidth to be compressed to the desired pulsewidth utilizing a predetermined temporal compression ratio (e.g. 1 ns to 1 ps compression); thus the system with a pulse stretched to two times the normal compression ratio can achieve output powers with double the repetition rate/average power or, by stacking the pulses in time, double the pulse energy; as compared to the normal systems using the same amplifiers. This technique can extends to a large number of pulse splittings, however there is a system level balance between generating and maintain the required bandwidth to enable the compression of the individual pulses with a predetermined temporal compression ratio.

In another embodiment, pulses from a common pulse generator are inputted into multiple amplifiers and the optical path lengths are controlled to provide precision timing between pulses; controlling path lengths by adjusting length of optical fiber in the low power portions of the system between pulse generator and different amplifiers can give nanosecond or even picosecond timing of output pulses; it also avoids a pulse during the plume of a previous ablation pulse (the plume can start as early as about 0.1 ns after an ablation pulse, and lasts about 10,000,000 ns, and can continue strong enough to cause significant inefficiency in the following pulse for about 1,000 ns). Picosecond spacing of pulses from different amplifiers can provide significantly more efficient machining. Essentially this can be using parallel amplifiers effectively.

In some embodiments, the pulses from different amplifiers are spaced in time, whereby a higher repetition rate is produced than a single amplifier system. With the at least two of the pulses from different amplifiers are spaced in time by less than 20 or 30 picoseconds for more efficient machining than pulses from different amplifiers are spaced in time by more than e.g. 40 picoseconds.

In some embodiments, the initial pulse is split by color to allow stacking of output pulses from the first and second amplifiers for producing system output pulse peak power higher than the amplifier output pulses. In some embodiments, the generating, amplifying and compressing are done within a man-portable system.

Multi-amplifier ablation systems can be a very important, especially when used in conjunction with an expensive automated production system; e.g., in a two amplifier system, the production rate can be essentially doubled, while the cost of the expensive automated production system remains the essentially same, as do some parts of the laser system.

The main operating costs that are doubled are the amplifier maintenance, material, and the electricity, however labor is not and thus operating cost per unit produced is decreased.

The capital cost per unit produced is even more significantly decreased.

The following is a comparison of capital cost of system using multi-amplifier capacities to cost of a single amplifier system in a hypothetical ablation system used with an automated production system.

Basic parts of laser system components: Pulse generator, Amplifier, and Compressor. Cost of a typical automated production system assumed to be $200,000. Cost of single amplifier laser system, assumed to be $200,000.

Cost of adding another amplifier, including its pump diodes and power supplies, as well as an optical splitter and an optical combiner, assumed to be $150,000.

Capital cost per part of single system assumed to be 16 cents (1 part/minute—written off over 5 years). Labor cost per part of single system assumed to be 6 cents machine operators, plus 6 cent others. Material and other costs proportional to number of parts assumed to be $0.10 per part.

Thus in this hypothetical, capital cost per part, labor cost per part, and costs proportional to number of parts are respectively are $0.16; $0.12; and $0.10 for a single amplifier system for a total of $0.38 per part.

Capital cost per part, labor cost per part, and costs proportional to number of parts are respectively are $0.11; $0.06; and $0.10 for a dual amplifier system for a total of $0.27 per part.

Capital cost per part, labor cost per part, and costs proportional to number of parts are respectively are $0.07; $0.03; and $0.10 for a four amplifier system for a total of $0.20 per part.

Capital cost per part, labor cost per part, and costs proportional to number of parts are respectively are calculated to be $0.0425; $0.0150; and $0.1000 for an eight amplifier system for a total of $0.1575 per part.

The following is an Example with an 8 color 6 amplifier system.

All splitters are dichroic; all amplifiers, delays, and combiners are preferably fiber; all input delays are preferably trimable. All pulse path lengths are preferably controlled. A 100 fs pulse with 20 nm of bandwidth/color at 1550 nm is preferably generated by a pulse generator, such as a AM modelocked laser diode for a 1550 nm emission.

### First Pulse in this Example

Amplifier One

A 100 fs pulse with 20 nm of bandwidth/color at 1550 nm is stretched and amplified to 4 ns pulse using single mode fiber and 100 mW EDFA. The 4 ns long pulse is sent into a series of amplifiers. Each EDFA has a gain flattening filter at the entrance of the amplifier. The gain flattening filter is used to maintain the compressible bandwidth to a maximum in this case 20 nm as the pulse is amplified. Initially the pulse is amplified with a single mode amplifier until the pulse reaches a peak power of ≧225 kW or where the peak power induces nonlinearities in the core of the gain fiber. The optical Kerr effect in silica drives phenomena such as four-wave mixing, self-phase modulation, Raman modulation, instability and solution formation. The 8 ns long pulse is delivered to a series of amplifiers with increasing diameters to keep the peak power below the 25 kW level. The energy in a single mode 7 μm core fiber is on the order of 10 μJ in a 1 ns pulse. For a 4 ns pulse the energy would need to be >80 μJ before inducing the nonlinearities. Stretching the pulse to longer pulse length has tremendous advantages in storing energy in the pulse and reducing the cost of the amplifiers. The peak power scales with the core diameter of the gain fiber. A 50 μm diameter core fiber with a 4 ns pulse will support an energy of ˜250 μJ, Utilizing pulse shaping to compensate for nonlinearities will enable a 4 ns pulse to achieve a 1 mJ energy for the fiber based system. The unfortunate aspect of this pulse duration/length >1 ns requires a compression grating that is not currently manufacturable. A solution is to implement a folded compressor structure that uses multiple interactions with the compression grating and a number of mirrors. The losses are accumulative hence 6 hits on a mirror with 96% reflectivity will have a net efficiency/reflectivity of (0.90)¹⁰ or ˜35%. This does not include the efficiency from multiple reflections from the compression grating. In general, with a pulse as long as 8 ns the loss is ˜95% of the energy. To eliminate this issue requires the implementation of high efficiency narrow band dichroic mirrors. Dichroic mirrors can be made to reflect a narrow spectrum/bandwidth and transmit the remainder of the bandwidth of the pulse. Dichroic mirrors can also be made with the opposite design. At 1500 nm center wavelength with a compressible bandwidth/color of 5 nm enables a compressed pulse of on the order of 750 fs. As such the 4 ns pulse can be separated into 4 separate 1 ns pulses each with minimal losses (˜5%). Each 1 ns pulse can be placed into single pass grating compressor with an optimal efficiency of 65%. A 1 ns pulses can be arranged to overlap temporally to make a single pulse with high energy or place into the compressor sequentially. The sequentially arranged pulses can have a separation of a few picoseconds to nanoseconds. Larger spacing between the sequentially arranged pulses is possible but larger spacing can create an unacceptable optical length with ins separation requiring a spacing of a ˜one foot.

Large stretched pulses are possible with greater bandwidth/color generation. To date at 1500 nm published data shows generation of 50 nm of bandwidths. At different wavelengths gasses and other media have been used to create an octave (1,000 nm) of bandwidth via super continuum generation. This approach has been use by researchers to produce pulse compressed to the sub femtosecond level.

A wavelength of 1 μm requires a bandwidth of a few nm to achieve a compressed pulse of 750 fs. As 20 nm bandwidth can produce 10 compressible pulses with 1 ns pulse lengths. So for a wavelength of a Nd-Yt fiber amplifier with a center wavelength of 1 μm one can stretch the pulse to 10 ns and then use high efficiency dichroic optics/filters to separate the 10 ns pulse into 10, 1 ns pulses and then use a single compression stage optimizing the energy extraction from the amplifier, optimizing efficiency of the compressor stage, maximizing the energy per pulse, minimizing the cost and size of the system.

As used herein, the term “ablation pulse” means an ultra-short a laser pulse having peak power which causes non-linear multi-photon ionization when the strong electrical field generated by the laser light is sufficient to remove electrons from the bulk material, resulting in the material vaporizing and being ejected in an ionization plume.

Ablative material removal previously has often been done using non-portable systems with optical benches weighing perhaps 1,000 pounds and occupying about 300 cubic feet.

In some embodiments, the ablation system is man-portable and includes a wheeled cart or a backpack. As used herein, the term “man-portable” means a system utilizing an optical amplifier that is an optically-pumped-amplifier with components that can be positioned by one man (e.g., in a rack, as opposed to being mounted on a optical bench weighing hundreds of pounds), regardless of whether the system is designed to be easily moved or not. In some embodiments, the man-portable units include a handheld probe.

The said U.S. Pat. No. 7,361,171 by Stoltz et al., is hereby incorporated by reference herein.

Although the present invention and its advantages have been described above, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification, but only by the claims.

What is claimed is:

1. A method of ablative material removal from a body, comprising:
generating an initial pulse in a pulse generator;
amplifying the initial pulse using an optically-pumped, optical amplifier within a portable system to generate an amplified pulse;
splitting the amplified pulse into at least two split pulses having different optical paths, and controlling optical path lengths of said at least two split pulses to determine a temporal relationship between said two split pulses;
reorienting said at least two split pulses onto a common path in air;
compressing the at least two split pulses using at least one air-path between gratings compressor to produce at least one ablation pulse output; and
applying the at least one compressed optical ablation pulse to the body, wherein the generating, amplifying and compressing steps are performed within the portable system.

2. The method of claim 1, wherein said reorienting said at least two split pulses onto a common path in air is done using a dichroic mirror.

3. The method of claim 2, wherein said splitting of said amplified pulse into at least two split pulses is done using a dichroic mirror.

4. The method of claim 1, wherein said ablation pulse is less than one picosecond in duration

5. The method of claim 1, wherein said at least two split pulses are overlapped during reorienting to produce a single ablation pulse.

6. The method of claim 1, wherein said at least two split pulses are offset-overlapped during reorienting to produce a two ablation pulses.

7. A method of ablative material removal from a surface of a body, comprising:
generating initial pulses in a pulse generator;
obtaining at least two pulses having different wavelengths into different optical paths;
amplifying the obtained pulses using separate optically-pumped optical amplifiers to generate separate amplified pulses;
reorienting said separate pulses onto a common path in air using a dichroic mirror, while controlling optical path lengths of said separate pulses to determine a temporal pulse relationship to form at least one reoriented pulse;
Then compressing the at least one reoriented pulse within the portable system using an air-path between gratings compressor, to generate at least one compressed optical ablation pulse; and
applying the compressed optical pulse to the surface of the body, wherein the generating, amplifying and compressing steps are performed within the portable system.

8. The method of claim 7, wherein said at least two split pulses are overlapped during reorienting to produce a single ablation pulse.

9. The method of claim 7, wherein said at least two split pulses are offset-overlapped during reorienting to produce a two ablation pulses.

10. The method of claim 7, wherein said splitting of said amplified pulse into at least two split pulses is done using a dichroic mirror.

11. A method of ablative material removal from a body, comprising:
generating an initial pulse in a pulse generator;
amplifying the initial pulse using of two optical amplifiers in series within a portable system, wherein two amplifiers are used to generate an amplified pulse, wherein the first amplifier is an optically pumped fiber-amplifier and a second amplifier is a Raman amplifier;
then splitting the amplified pulse into at least two split pulses having different optical paths, and controlling optical path lengths of said at least two split pulses to determine a temporal relationship between said two split pulses;
then reorienting said at least two split pulses on a common path in air;
compressing the at least two split pulses using at least one air-path between gratings compressor to produce at least one ablation pulse output; and
applying the at least one compressed optical ablation pulse to the body, wherein the generating, amplifying and compressing steps are performed within the portable system, and wherein optical path length between said initial pulse splitter and said reorienting is controlling to determine a temporal relationship at least two split pulses, whereby at least one of pulse energy and repetition rate can be at least substantially doubled.

12. The method of claim 1, wherein the generating, amplifying and compressing are done within a man-portable system.
